(19) United States
(12) Patent Application Publication
(10) Pub. No.: US 2015/0351609 A1
(43) Pub. Date: Dec. 10, 2015

(54) BENDING OPERATION APPARATUS FOR ENDOSCOPE AND ENDOSCOPE

(71) Applicant: SG ENDOSCOPY PTE. LTD., Singapore (SG)

(72) Inventor: Jin Mei Fan, Singapore (SG)

(73) Assignee: SG ENDOSCOPY PTE. LTD., Singapore (SG)

(21) Appl. No.: 14/760,812

(22) PCT Filed: Jan. 14, 2014
(86) PCT No.: PCT/SG2014/000012

§ 371 Date: Jul. 14, 2015

(30) Foreign Application Priority Data
Jan. 14, 2013 (SG) 201300283-7

(51) Int. Cl. A61B 1/005

(52) U.S. Cl. CPC A61B 1/0055; A61B 1/0057; A61B 1/0052

(57) ABSTRACT

In a bending operation apparatus provided with pinions, and two pairs of rack members connected to two pair of operation wires arranged to extend up to a bending part, where operation wires are arranged in tubular covering members, bend-preventing members which cover the operation wires in an extendable and contractable manner in driving directions of the operation wires to prevent bends of the operation wires are provided between joint portions of the operation wires with the rack members and a covering member fixing part. Further, moving ranges of the rack members are set to be changeable. Furthermore, covering member holding parts are arranged on an inner peripheral face of a connection tube part of the bending operation apparatus.

### TECHNICAL FIELD

The present invention relates to a bending operation apparatus which bends a bending part formed at a distal end portion of an insertion part of an endoscope to a desired state, and an endoscope provided with the bending operation apparatus.

### BACKGROUND ART

An endoscope is configured by disposing a bending part which is bendable at a distal end portion of a hollow insertion part configured to be flexible, and it is put in a desired bent state by operating a bending operation apparatus arranged at a base portion of the insertion part. In such an endoscope, the bending part is configured to be bendable in two directions orthogonal to each other on a plane perpendicular to an axial direction of the insertion part, for example, in a vertical direction (UD: upward and downward directions) and in a horizontal direction (RL: rightward and leftward directions).

In this case, two sets of operation wires pairs composed of two operation wires corresponding to the vertical direction and two operation wires corresponding to the horizontal direction, respectively, are arranged between the bending part of the endoscope and the bending operation apparatus, so that such an operation that one of operation wires of each operation wire pair is pulled in and the other thereof is pushed out is performed in the bending operation apparatus.

As the bending operation apparatus performing pushing-out and pulling-in of the operation wire pairs in this manner, there is a bending operation apparatus provided with a pulley, where a pair of operation wires, both ends of each operation wire being coupled to each other, are wound on the pulley, and the operation wires on both sides are pushed out and pulled in by rotationally driving the pulley (see Patent Literature 1).

Further, as a bending operation apparatus provided with another configuration, there is a bending operation apparatus provided with a pinion, where racks connected to base sides of a pair of operation wires, respectively, are caused to mesh with both sides of the pinion, and the operation wires on both sides are pushed out and pulled in by rotationally driving the pinion (see Patent Literature 2).

Now, in such a bending operation apparatus for an endoscope, such a case may occur that, when bending operation of the bending part is performed, the operation wire is loosened or flexed due to an operation state, so that the operation wire cannot be operated as an operator's intention, which does not result in achievement of a desired bending angle of the bending part.

Further, the maximum bending angle of the bending part is determined according to a pushing-out amount of the operation wire and a pulling-in amount thereof. In this case, when the bending part is configured so to bend in the horizontal direction and the vertical direction, regarding the adjustment of the pushing-out amounts and the pulling-in amounts of the operation wires, the pair of operation wires for the vertical direction and the pair of operation wires for the horizontal direction must be individually performed, respectively, which results in much complexity (see Patent Literature 3).

Further, an endoscope configured such that a cover member which has an approximately parallelepiped shape as a whole and in which four stoppers disposed along an axial direction within a case and fixed therein with two operation wires for a horizontal direction and two operation wires for a vertical directions for restricting moving ranges of the respective operation wires are extended is provided so that the four stopper members can be collectively adjusted substantially at one spot has also been proposed.

However, in such an endoscope, there is such a drawback that the number of constituent parts increases, a manufacturing cost becomes expensive, and the weight of an operation part also increases, which results in burden for an operator.

### PRIOR ART LITERATURE

### Patent Literature

[Patent Literature 1] Japanese Patent Application Laid-Open No. H07-27984

[Patent Literature 2] Japanese Patent Application Laid-Open No. 2009-225876

[Patent Literature 3] Japanese Patent No. 4668723

### SUMMARY OF INVENTION

### Problem to be Solved by Invention

The present invention has been made in view of the above-described circumstances, and an object thereof is to provide a bending operation apparatus for an endoscope where operation wires for operating a bending part can be operated properly according to an operator's intention, adjustment of an bending angle of the bending part can be performed easily, and the number of parts is reduced, which results in decrease of a manufacturing cost, and an endoscope.

### Means for Solving the Problem

According to a first aspect of the present invention for solving the above problem, there is provided a bending operation apparatus for an endoscope which includes: a case; an operation member arranged outside the case; a pinion arranged within the case and rotationally driven by the operation member; and two rack members arranged so as to sandwich the pinion diametrically, meshing with the pinion, and connected to a pair of two operation wires arranged to extend up to a bending part arranged in a bendable fashion at a distal end of an insertion part inserted into a body to be examined, where the operation wires are fixed at end portions thereof within the case, are arranged within tubular covering members arranged in the insertion part, and drive the bending part in a bending fashion by operation of the operation member, wherein bend-preventing members which cover circumferences of portions of the operation wires included in the case in an extendable and contractable manner in driving directions of the operation wires to prevent bends of the operation wires are arranged, the portions of the operation wires being positioned between joint portions of the operation wires with the rack members and fixing portions thereof to the covering members.

According to the first aspect of the present invention, loosening or bending of the operation wires within the operation apparatus can be prevented by the bend-preventing members.

According to a second aspect of the present invention, there is provided the bending operation apparatus for an endoscope according to the first aspect, wherein the bend-preventing members are coil-like members wound coarsely.

According to the second aspect of the present invention, since the bend-preventing members are composed of the coil-like members wound coarsely, the configuration of the bending operation apparatus for an endoscope can be made simple.

According to a third aspect of the present invention, there is provided the bending operation apparatus for an endoscope according to the first or second aspect, wherein the bend-preventing members are made of metal.

According to a third aspect of the present invention, since the bend-preventing members are made of metal, the bending operation apparatus for an endoscope can be configured to have a high strength and a high durability.

According to a fourth aspect of the present invention, there is provided a bending operation apparatus for an endoscope which includes: a case; an operation member arranged outside the case; a pinion arranged within the case and rotationally driven by the operation member; and two rack members arranged so as to sandwich the pinion diametrically, meshing with the pinion, and connected to a pair of two operation wires arranged to extend up to a bending part arranged in a bendable fashion at a distal end of an insertion part inserted into a body to be examined, where the operation wires are fixed at end portions thereof within the case, are arranged within tubular covering members arranged in the insertion part, and drive the bending part in a bending fashion by operation of the operation member, wherein the bending operation apparatus for an endoscope further includes a moving range setting means for determining moving ranges of the two rack members to the side of the insertion part collectively, the moving range setting means being disposed within the case such that a mounting position thereof can be changed.

According to the fourth aspect of the present invention, since the positions of the fixing portions of the covering members as a moving range setting means are made changeable, a drive-enabling range of the set of operation wires which drive the bending part, namely, the bending range of the bending part can be easily changed by the position adjustment of the fixing portions of the covering members. Thereby, even an endoscope having a different specification, namely, having a different bending angle can be accommodated by changing the positions of the fixing portions of the covering members in the same bending operation apparatus. Therefore, it is unnecessary to adjust the respective operating wires of the pair of operating wires for a vertical direction and the pair of operating wires for a horizontal direction individually in the conventional manner.

According to a fifth aspect of the present invention, there is provided the bending operation apparatus for an endoscope according to the fourth aspect, wherein a plurality of pairs of operation wires for bending the bending part in different directions is provided, and the moving range setting means is disposed so as to correspond to each of the operation wires constituting the pair.

According to the fifth aspect of the present invention, the drive ranges of the bending part in two directions (a horizontal direction and a vertical direction) can be changed easily by the position adjustment of the fixing part of the covering members, and it is unnecessary to adjust the respective operating wires of the pair of operating wires for a vertical direction and the pair of operating wires for a horizontal direction individually like the fourth aspect.

According to a sixth aspect of the present invention, there is provided the bending operation apparatus for an endoscope according to the fourth or fifth aspect, wherein the moving range setting means is screwed within the case such that a fixing position of the moving range setting means can be changed. According to the sixth aspect, the position of the fixing part of the covering members can be changed easily according to tightening and releasing operations of the screw of the fixing device of the covering members.

According to a seventh aspect of the present invention, there is provided a bending operation apparatus for an endoscope which includes: a case; an operation member arranged outside the case; a pinion arranged within the case and rotationally driven by the operation member; and two racks arranged so as to sandwich the pinion diametrically, meshing with the pinion, and connected to a pair of two operation wires arranged to extend up to a bending part arranged in a bendable fashion at a distal end of an insertion part inserted into a body to be examined, where the operation wires are fixed at end portions thereof within the case, are arranged within a tubular covering members arranged in the insertion part, and drive the bending part in a bending fashion by operation of the operation member, wherein a cylindrical connection tube part is provided between the case and the insertion part, and a covering member insertion part for arranging the covering members along an inner face portion of the connection tube part is provided on the inner face portion of the connection tube part.

According to the seventh aspect, since the covering tubes having the operation wires inserted therein can be fixed by the covering member insertion part provided at the connection tube part, a plurality of covering tubes are prevented from intersecting with each other or coming in contact with each other, so that operations of the operation wires can be performed smoothly.

According to an eighth aspect of the present invention, there is provided the bending operation apparatus for an endoscope according to the seventh aspect, wherein the covering member insertion part is an annular member formed on the inner face portion of the connection tube part integrally with the connection tube part.

According to the eighth aspect, since the covering member insertion part is provided on the side close to the bending operation apparatus, such an event can be prevented effectively that the covering members tangle mutually.

Further, since the fixing part is formed integrally with the connection tube part as the annular member, it is unnecessary to attach a covering member insertion part prepared as a separate member to the connection tube part, which can result in simple configuration and easy assembling work.

According to a ninth aspect of the present invention, there is provided the bending operation apparatus for an endoscope according to the seventh or eighth aspect, wherein the connection tube part is provided with a forceps insertion port for inserting a forceps into the insertion part.

According to the ninth aspect, when a forceps is inserted from the forceps insertion port, the forceps can be inserted smoothly without being blocking by the covering members into which the operation wire has been inserted.

According to a tenth aspect of the present invention, there is provided an endoscope including: the bending operation apparatus for an endoscope according to any one of the first to ninth aspects; and an observing means for observing inside of a body to be examined from the bending part.

According to the tenth aspect, the operation wires for operating the bending part can be correctly operated according to an operator's intention, and the endoscope can be operated successfully.

### Effect of Invention

According to the present invention, a bending operation apparatus for an endoscope where the operating wires for operating the bending part can be operated properly according to an operator's intention, adjustment of the bending angle of the bending part can be performed easily, and the number of parts is decreased, which can result in reduction of a manufacturing cost, and an endoscope can be provided.

### BRIEF DESCRIPTION OF THE DRAWINGS

FIG. 1 is a perspective view showing an endoscope according to an embodiment of the present invention;

FIG. 2 is a perspective view showing an operation part of the endoscope according to the embodiment of the present invention;

FIG. 3 is a perspective view showing an internal structure of a bending operation apparatus of the operation part of the endoscope according to the embodiment of the present invention;

FIGS. 4A and 4B show an internal structure of the bending operation apparatus, FIG. 4A being a plan view showing a UD bending operation part, and FIG. 4B being a plan view showing an RL bending operation part;

FIGS. 5A to 5C show operations of the UD bending operation part, FIG. 5A being an illustrative view showing an neutral position when a covering member fixing portion is disposed at a first position, FIG. 5B being an illustrative view showing a maximum operation position of a rack member when the covering member fixing portion is disposed at the first position, and FIG. 5C being an illustrative view showing a maximum operation position of the rack member when the covering member fixing portion is disposed at a second position;

FIGS. 6A to 6C show operations of the RL bending operation part, FIG. 6A being an illustrative view showing a neutral position when a rack stopping member is disposed at a first position, FIG. 6B being an illustrative view showing a maximum operation position of the rack member when the rack stopping member is disposed at the first position, and FIG. 6C being an illustrative view showing a maximum operation position of the rack member when the rack stopping member is disposed at a second position;

FIG. 7 is a perspective view showing the bending operation apparatus and a jump state of an operation wire;

FIG. 8 is a perspective view showing a connection tube part of the bending operation apparatus; and

FIG. 9 is a sectional view of the connection tube part taken along line A-A in FIG. 8.

### EMBODIMENT FOR CARRYING OUT THE INVENTION

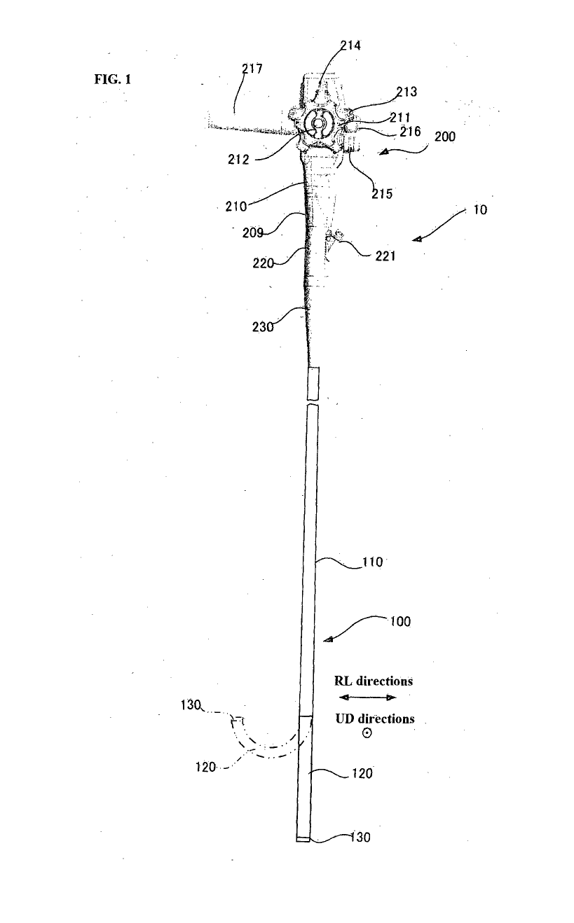

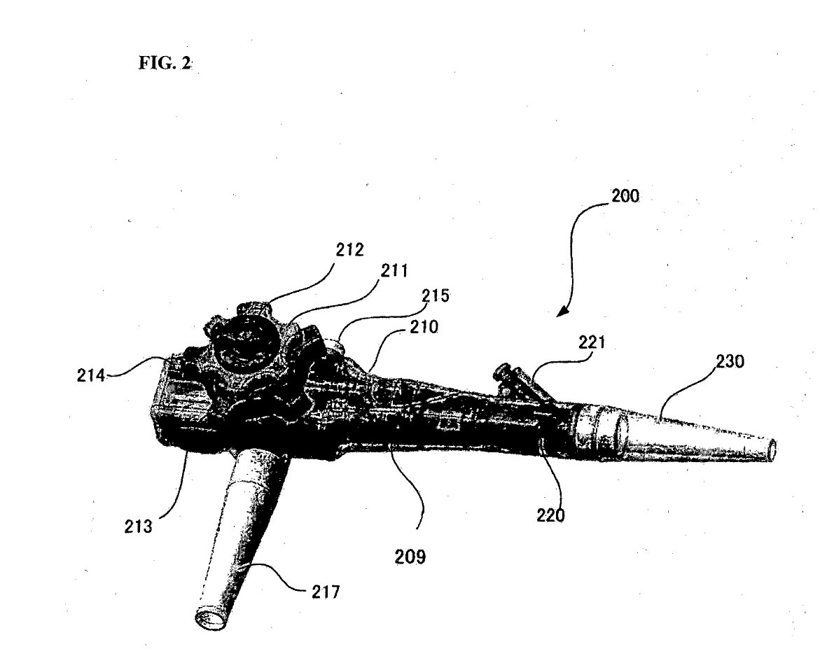

A bending operation apparatus for an endoscope and an endoscope according to an embodiment for carrying out the invention (hereinafter called “embodiment” simply) will be described. FIG. 1 is a perspective view showing an endoscope according to an embodiment of the present invention, and FIG. 2 is a perspective view showing a bending operation apparatus for an endoscope according to the embodiment of the present invention.

As shown in FIG. 1, an endoscope 10 according to this embodiment is provided with an insertion tube part 100 to be inserted into a body to be examined and a bending operation apparatus 200 which is disposed at a proximal end portion of the insertion tube part 100 and by which an operator performs various operations including a bending operation. The insertion tube part 100 is provided with an insertion part 110 having flexibility, a bending part 120 disposed at a distal end portion of the insertion part 110 and bent according to an operation on hand of the bending operation apparatus 200, and a distal end part 130 disposed at a distal end of the bending part 120 and including not only an image sensor (not shown), an optical system for imaging (not shown), and an optical system for illumination (not shown) but also various members.

The bending part 120 is configured so as to be bent and deformed freely according to operations of operation wires (not shown) inserted in the insertion tube part 100 from the bending operation apparatus 200 up to the bending part 120 (a bent state is shown by an imaginary line in FIG. 1).

In the insertion tube part 100 according to the embodiment, two sets of operation wires paired, namely, a total of four operation wires are arranged between the bending operation apparatus 200 and the bending part 120, and the bending part 120 can be bent according to a hand operation of the bending operation apparatus 200 in two directions orthogonal to each other on a plane perpendicular to an axial direction of the insertion part 110, for example, in a horizontal direction (RL directions: right and left directions in FIG. 1) and in a vertical direction (UD directions: in upward and downward directions in FIG. 1).

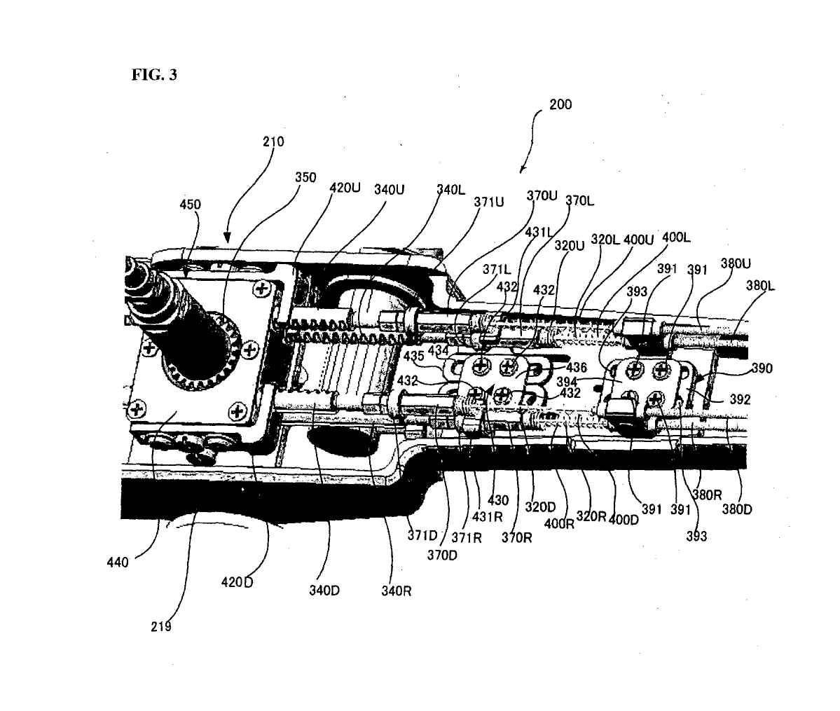

As shown in FIG. 2 and FIG. 3, the bending operation apparatus 200 is composed of a bending operation apparatus main body 210 provided with a case 209, a connection tube part 220 connecting the bending operation apparatus main body 210 and the insertion part 110 with each other, and a taper part 230. The bending operation apparatus main body 210 includes, in the case 209, a UD operation handle 213 which is an operation part for operating operation wires 320U and 320D in the UD directions, a lock handle 214 fixing the UD operation handle 213, an RL operation handle 211 which is an operation part for operating operation wires 320R and 320L in the RL directions, a lock handle 212 fixing the RL operation handle 211, a gas-feeding/water-feeding valve 215 for feeding air or water to the distal end part 130, a suction valve 216 for suctioning gas or liquid from the distal end part 130, and a light guide 217 connected to an image display apparatus (not shown), a CCD camera (not shown), and an illumination apparatus(not shown). Further, the connection tube part 220 is provided with a forceps insertion port 221.

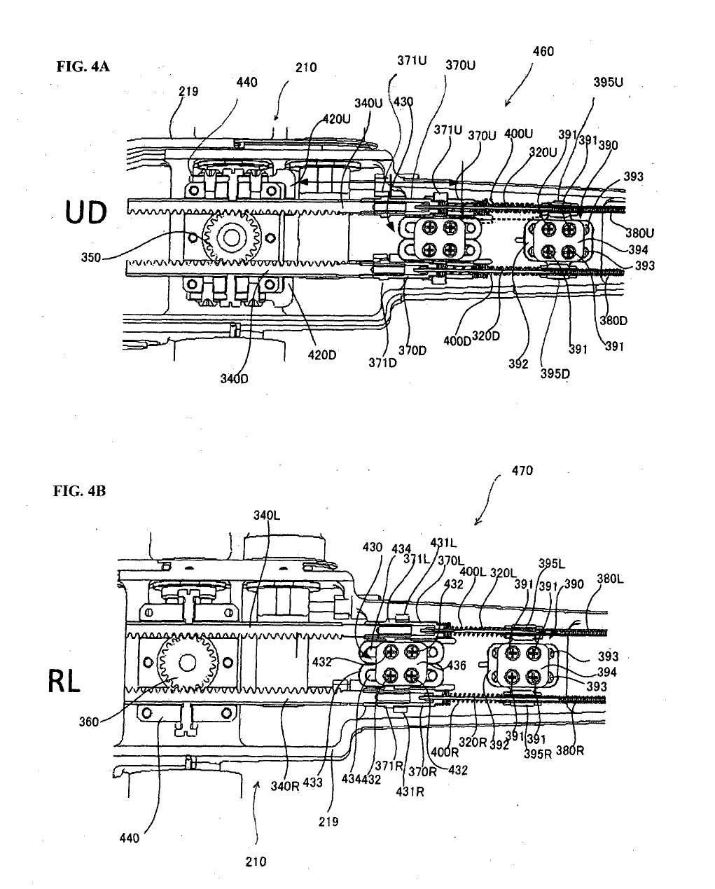

Next, an internal mechanism of the bending operation apparatus 200 will be described. FIG. 3 is a perspective view showing an internal structure of the bending operation apparatus 200 according to the embodiment, and FIGS. 4A and 4B show an internal structure of the bending operation apparatus 200, FIG. 4A being a plan view showing a UD (upward and downward) bending operation part 460 and FIG. 4B being a plan view showing a RL (right and left) bending operation part 470.

Distal end portions of four operation wires 320U, 320D, 320R, and 320L disposed to extend up to the bending part 120 are arranged in the bending operation apparatus main body 210. Two operation wires 320U and 320D of the four operation wires are paired, and when an operation is made such that one of the two operation wires 320U and 320D is pulled in while the other is pushed out, the bending part 120 is bent in one direction of the RL directions. Further, two operation wires 320R and 320L are paired to be disposed below the operation wires 320U and 320D, and when an operation is made such that one of the two operation wires 320R and 320L is pulled in while the other is pushed out, the bending part 120 is bent in one direction of the RL directions.

Rack members 340U and 340D are connected to end portions of the operation wires 320U and 320D. The rack members 340U and 340D are arranged so as to sandwich a pinion 350 arranged in the bending operation apparatus 200 in a diametrical direction of the pinion 350 and mesh with the same. The operation wires 320U and 320D, and the rack members 340U and 340D are connected by connection members 370U and 370D provided with flange portions 371U and 371D, respectively.

Further, rack members 340R and 340L are connected to end portions of the operation wires 320R and 320L. The rack members 340R and 340L are arranged to sandwich a pinion 360 arranged in the bending operation apparatus 200 in a diametrical direction of the pinion 360 and mesh with the same. The operation wires 320R and 320L, and the rack members 340R and 340L are connected by connection members 370R and 370L provided with flange portions 371R and 371L, respectively.

The pinion 350 for UD direction operation and the pinion 360 for RL direction operation are arranged coaxially with each other within a gear box 440. In this embodiment, the pinion 350 is disposed above the pinion 360. The pinion 350 is rotationally driven by the UD operation handle 213, while the pinion 360 is rotationally driven by the RL operation handle 211. The pinion 350 and the UD operation handle 213, and the pinion 360 and the RL handle 211 are coupled by a multi-shaft 450.

Here, the operation wires 320U, 320D, 320R, and 320L are arranged within the insertion tube part 100 in a state where they have been inserted into covering members 380U, 380D, 380R, and 380L, respectively. The covering members 380U, 380D, 380R, and 380L are members formed by winding wires in coil shapes densely. End portions of the covering members 380U, 380D, 380R, and 380L on the side of the bending operation apparatus main body 210 are fixed to the bending operation apparatus main body 210 at a covering member fixing part 390.

As shown in FIGS. 4A and 4B, the covering member fixing part 390 is provided with arm parts 395U, 395D, 395R, and 395L for fixing the covering members 380U, 380D, 380R, and 380L, respectively. The covering member fixing part 390 is fixed to the bending operation apparatus main body 210 such that an arrangement position of the covering member fixing part 390 can be changed along a driving direction within the bending operation apparatus main body 210. That is, the covering member fixing part 390 can be fixed by screws 391 after the position of a retainer plate 394 is adjusted along long holes 393 formed in a base portion 392. Thereby, the covering member fixing part 390 can be mounted on the bending operation apparatus main body 210 such that the position thereof can be changed along the axial direction.

The covering member fixing part 390 serves as a moving range setting means for determining moving ranges of the rack members 340U and 340D connected to the covering members 380U and 380D to the side of the insertion tube part 100 (a downstream side) in addition to fixing the covering members 380U, 380D, 380R, and 380L within the bending operation apparatus main body 210.

That is, when the connection members 370U and 370D are moved to the downstream side by driving the rack members 340U and 340D, the flange portions 371U and 371D come in contact with the arm parts 395U and 395D of the covering member fixing part 390 to restrict movements of the rack members 340U and 340D to the downstream side. Thereby, one of the moving ranges of the operation wires 320U and 320D is restricted.

Further, stoppers 420U and 420D are arranged in the gear box 440 around the pinion 350 and the pinion 360. When the connection members 370U and 370D are moved to an upstream side by driving the rack members 340U and 340D, the stoppers 420U and 420D abut on the flange portions 371U and 371D to stop the rack members 340U and 340D and restrict movements of the rack members 340U and 340D to the upstream side, thereby restricting the other of the moving ranges of the operation wires 320U and 320D.

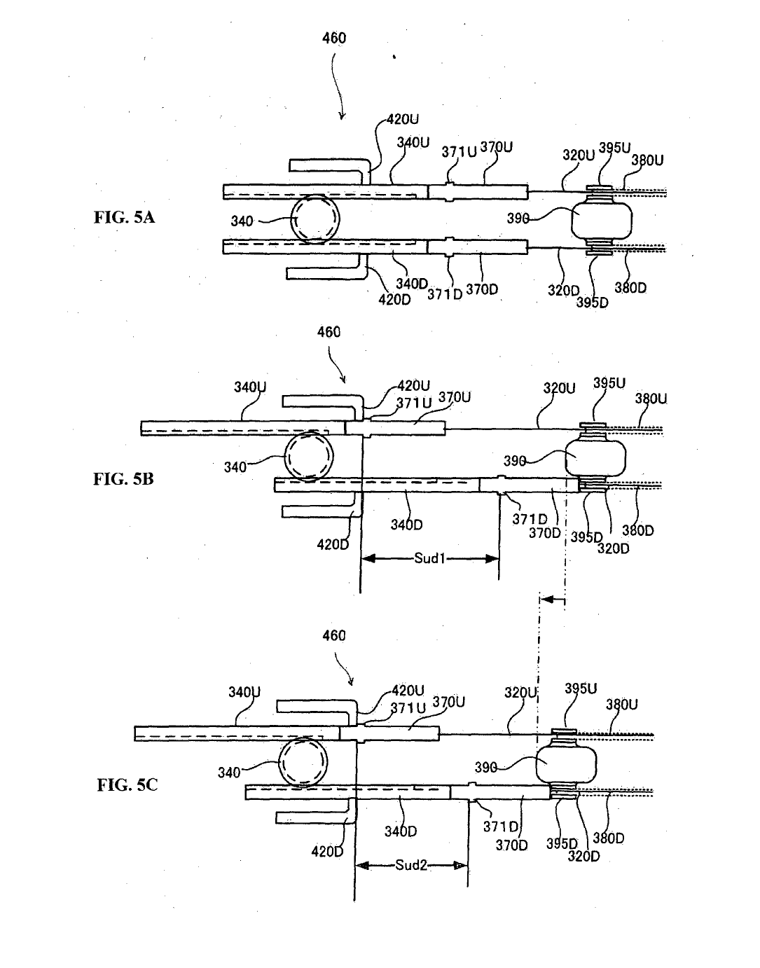

FIGS. 5A to 5C show operations of the UD bending operation part 460, FIG. 5A being an illustrative view showing a neutral position when the covering member fixing part 390 is disposed at a first position, FIG. 5B being an illustrative view showing a maximum operation position of a rack member when the covering member fixing part 390 is disposed at the first position, and FIG. 5C being an illustrative view showing a maximum operation position of the rack member when the covering member fixing part 390 is disposed at a second position.

When the covering member fixing part 390 is fixed at the first position shown in FIGS. 5A and 5B, the moving ranges of the rack members 340U and 340D become Sud1. Thereby, the maximum bending angle of the bending part 120 in the UD directions can be defined.

In this embodiment, the moving ranges of the rack members 340U and 340D can be changed by moving the covering member fixing part 390 along the long holes 393 in the axial direction. For example, when the covering member fixing part 390 is disposed at a second position positioned on an upstream side from the first position, as shown in FIG. 5C, the moving ranges become small such as Sud2. Thereby, the maximum bending angle of the bending part 120 can be made small. Further, when the covering member fixing part 390 is moved to the downstream side beyond the first position, the maximum bending angle of the bending part 120 can be made large.

Further, as shown in FIG. 4B, in this embodiment, a rack stopping member 430 serving as a moving range setting means for determining the moving ranges of the rack members 340R and 340L to the side of the insertion tube part 100 (a downstream side) is disposed. The rack stopping member 430 is provided with a retainer plate 436 provided with bulging portions 431R and 431L coming in contact with the flange portions 371R and 371L of the connection members 370R and 370L.

Further, the rack stopping member 430 is fixed to the bending operation apparatus main body 210 by screws 432 such that the arrangement position thereof can be changed along the driving direction within the bending operation apparatus main body 210. The rack stopping member 430 can be fixed by the screws 432 after the position adjustment of a retainer plate 436 is performed along long holes 434 formed in a base portion 433. Thereby, the rack stopping member 430 can be mounted to the bending operation apparatus main body 210 such that the position thereof can be changed.

Further, in this embodiment, when the connection members 370R and 370L are moved to the upstream side by driving the rack members 340R and 340L, the flange portions 371R and 371L come in contact with the gear box 440 to stop.

In this embodiment, therefore, the moving ranges of the rack members 340R and 340L, namely, the moving ranges of operation wires 320R and 320L to the downstream side can be changed by moving the rack stopping member 430.

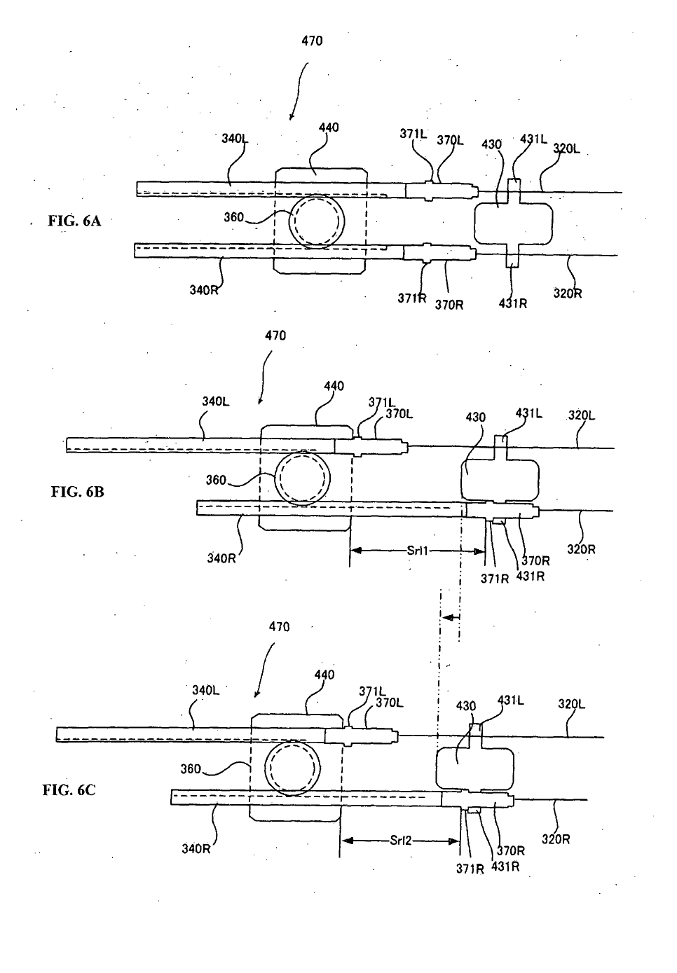

FIGS. 6A to 6C show operations of the RL bending operation part 470, FIG. 6A being an illustrative view showing a neutral position when the rack stopping member 430 is disposed at a first position, FIG. 6B being an illustrative view showing the maximum operation positions of the rack members 340R and 340L when the rack stopping member 430 is disposed at the first position, and FIG. 6C being an illustrative view showing the maximum operation positions of the rack members 340R and 340L when the rack stopping member 430 is disposed at a second position.

When the rack stopping member 430 is fixed at the first position shown in FIGS. 6A and 6B, the moving ranges of the rack members 340R and 340L become Srl1. Thereby, the maximum bending angle of the bending part 120 can be defined.

In this embodiment, the moving ranges of the rack members 340R and 340L can be changed by moving the rack stopping member 430. For example, as shown in FIG. 6C, when the rack stopping member 430 is disposed at the second position to the upstream side beyond the first position, the moving ranges of the rack members 340R and 340L become small such as Srl2. Thereby, the maximum bending angle of the bending part 120 can be made small. Further, when the rack stopping member 430 is moved to the downstream side beyond the first position, the maximum bending angle of the bending part 120 can be made large.

Thus, in the endoscope 10 according to this embodiment, since the maximum bending angle of the distal end part 130 can be changed, the bending operation apparatus 200 having the same structure can be applied to an endoscope of a different specification provided with a bending part with a different maximum bending angle by only changing the mounting positions of the covering member fixing part 390 and the rack stopping member 430. Incidentally, the position adjustments of the covering member fixing part 390 and the rack stopping member 430 in the bending operation apparatus 200 can be performed in a manufacturing stage of the bending operation apparatus 200.

Further, in the bending operation apparatus 200 according to the embodiment, bend-preventing members 400U, 400D, 400R, and 400L which cover outer peripheries of the operation wires 320U, 320D, 320R, and 320L in an extendable and contractable manner in driving directions of the operation wires 320U, 320D, 320R, and 320L to prevent bends of the operation wires 320U, 320D, 320R, and 320L are arranged between the rack members 340U, 340D, 340R, and 340L of the operation wires 320U, 320D, 320R, and 320L and the covering members 380U, 380D, 380R, and 380L fixed at the covering member fixing part 390.

The bend-preventing members 400U, 400D, 400R, and 400L are coil members made of metal and wound coarsely. Therefore, the configurations of the bend-preventing members 400U, 400D, 400R, and 400L can be made simple and durable.

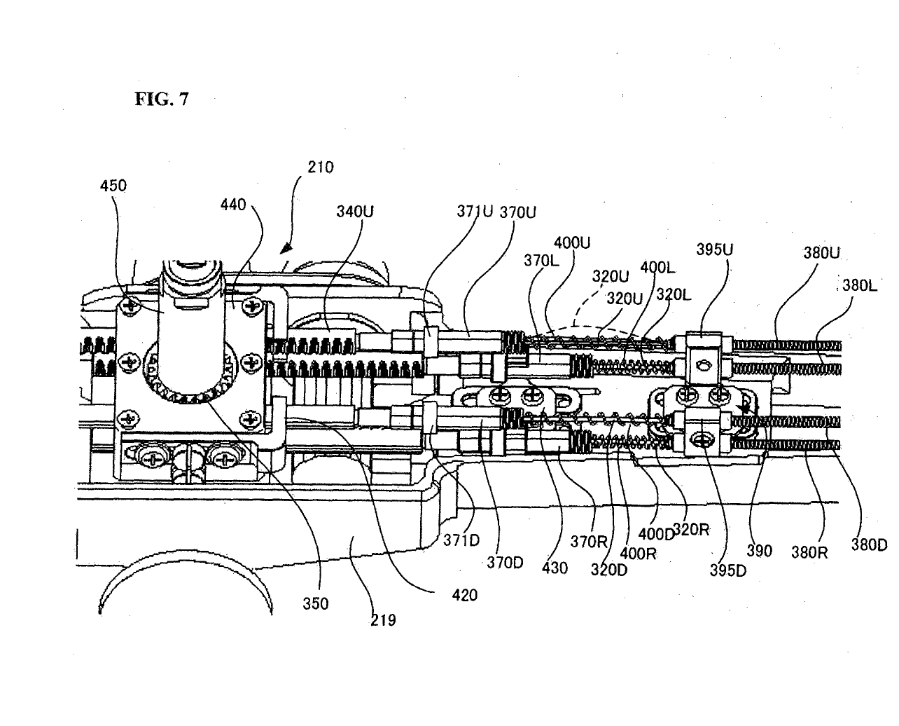

As shown in FIG. 7, end portions of the bend-preventing members 400U, 400D, 400R, and 400L which are positioned on the side of the rack members are fixed by winding the coil members on outer peripheries of end portions of the connection members 370U, 370D, 370R, and 370L connected and fixed to the rack members 340U, 340D, 340R, 340L, while end portions of the bend-preventing members 400U, 400D, 400R, and 400L which are positioned on the side of the covering members are fixed by winding the coil members on outer peripheries of end portions of the covering members 380U, 380D, 380R, and 380L.

In the endoscope 10 according to this embodiment, since the bend-preventing members 400U, 400D, 400R, and 400L are arranged around the operation wires 320U, 320D, 320R, and 320L, bends (wire jumps) of the operation wires 320U, 320D, 320R, and 320L generated when the operation wires 320U, 320D, 320R, and 320L are pushed out from such a state that the operation wires 320U, 320D, 320R, and 320L have been pulled in can be prevented, so that operations of the operation wires 320U, 320D, 320R, and 320L can be performed securely. Incidentally, in FIG. 7, a state where the operation wire 320U has been bent (wire-jumped) when the bend-preventing member 400U is not provided is shown by a broken line.

Incidentally, the bend-preventing members 400U, 400D, 400R, and 400L are only required to cover the peripheries of the operation wires 320U, 320D, 320R, and 320L in an extendable and contractable manner so as not to bend them without being required to impart biasing forces to the rack members 340U, 340D, 340R, and 340L. Therefore, as the bend-preventing members 400U, 400D, 400R, and 400L, bellows-like members can be used instead of the coil-like members.

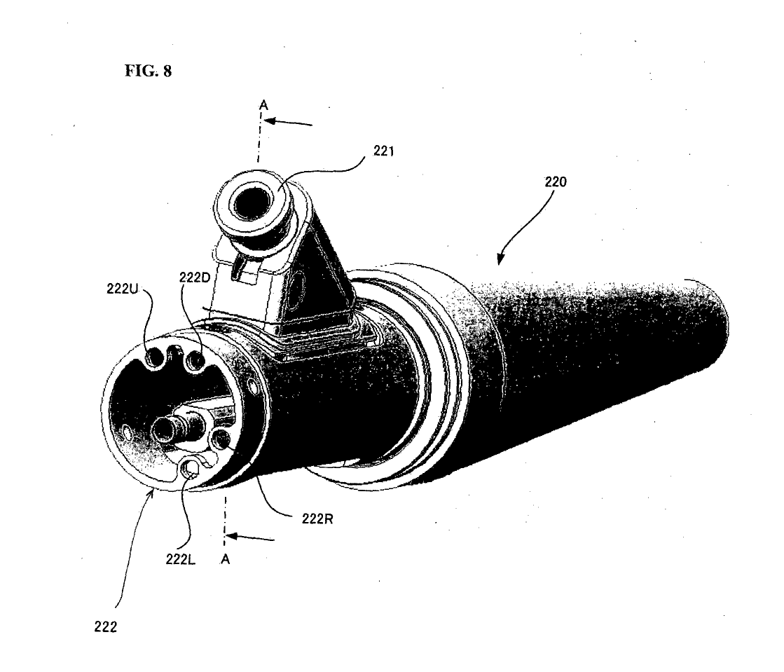

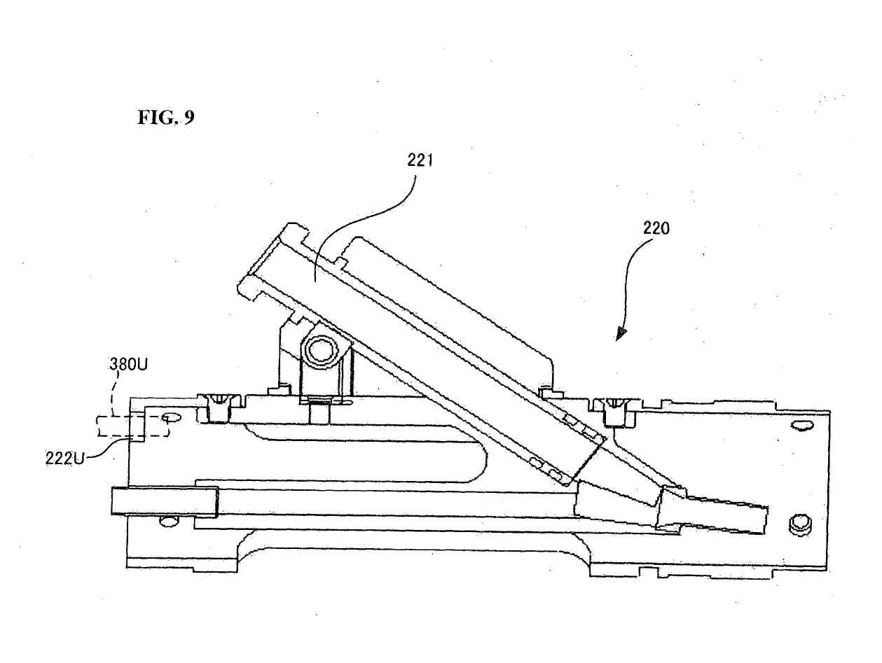

Next, the configuration of the connection tube part 220 will be described. FIG. 8 a perspective view showing a connection tube part of the bending operation apparatus, and FIG. 9 is a sectional view of the connection tube part taken along line A-A in FIG. 8. In the insertion tube part 100 of the endoscope 10 according to this embodiment, the connection tube part 220 is provided with the forceps insertion port 221 and further provided with covering member holding parts 222U, 222D, 222R, and 222L which hold the covering members 380U, 380D, 380R, and 380L having the operation wires 320U, 320D, 320R, and 320L inserted into the inner face portion of the connection tube part 220. The covering member holding parts 222U, 222D, 222R, and 222L are provided on an inner peripheral face portion of the annular member 222 formed on an inner periphery of the connection tube part 220 integrally with the connection tube part 220.

Therefore, since the covering members 380U, 380D, 380R, and 380L are inserted into the covering member holding parts 222U, 222D, 222R, and 222L corresponding thereto, respectively, to be fixed on an inner peripheral face of the connection tube part 220, operations of the operation wires 320U, 320D, 320R, and 320L can be prevented from being blocked due to contact with the covering members 380U, 380D, 380R, and 380L or tangling thereof, and other operations thereof can be prevented from being blocked.

Further, in this embodiment, since the annular member 222 which is the covering member insertion part is provided on the side close to the bending operation apparatus 200 of the endoscope 100, such an event can be prevented effectively that the covering members 380U, 380D, 380R, and 380L covering the operation wires 320U, 320D, 320R, and 320L tangle mutually.

Furthermore, since the covering member insertion part is formed integrally with the connection tube part 220 as the annular member 222, it is unnecessary to attach a covering member insertion part as a separate member via a connection member, which can result in simple configuration and easy assembling.

Further, when a forceps is inserted from the forceps insertion port 221 into the endoscope 10, the forceps can be inserted smoothly without being blocked by the covering members 380U, 380D, 380R, and 380L in which the operation wires 320U, 320D, 320R, and 320L have been inserted. The operation wires 320U, 320D, 320R, and 320L for operating the bending part 120 can be operated correctly according to an operator's intention, so that the endoscope can be operated excellently.

As described above, according to the endoscope 10 according to this embodiment, the operation wires 320U, 320D, 320R, and 320L for operating the bending part 120 can be operated correctly according to an operator's intention.

Incidentally, in this embodiment, the case where the bend-preventing member 400 is made of metal has been described as an example, but the bend-preventing member 400 is not limited to the above embodiment and it may be made of synthetic resin.

### INDUSTRIAL APPLICABILITY

Since the present invention is widely applied to the bending operation apparatus for an endoscope and an endoscope, it has an industrial applicability widely.

### EXPLANATION OF REFERENCE NUMERALS

10: endoscope
100: insertion tube part
110: insertion part
120: bending part
130: distal end part
200: bending operation apparatus
209: case
210: bending operation apparatus main body
211: RL operation handle (operation part)
212: lock handle
213: UD operation handle (operation part)
214: lock handle
215: gas-feeding/water-feeding valve
216: suction valve
217: light guide
220: connection tube part
221: forceps insertion port
222: annular member (covering member insertion part)
222U: covering member holding part
222D: covering member holding part
222R: covering member holding part
222L: covering member holding part
230: taper portion
320R: operation wire
320L: operation wire
320U: operation wire
320D: operation wire
340R: rack member
340L: rack member
340U: rack member
340D: rack member
350: pinion
360: pinion
370U: connection member
370D: connection member
370R: connection member
370L: connection member
371R: flange portion
371L: flange portion
371U: flange portion
371D: flange portion
380U: covering member
380D: covering member
380R: covering member
380L: covering member
390: covering member fixing part(moving range setting means)
391: screw
392: base portion
393: long hole
394: retainer plate
395U: arm part
395D: arm part
395R: arm part
395L: arm part
400U: bend-preventing member
420U: stopper
420D: stopper
430: rack stopping member
431R: bulging portion
432: screw
433: base portion
434: long hole
436: retainer plate
440: gear box
450: multi-shaft
460: UD bending operation part
470: RL bending operation part

What is claimed is:

1. A bending operation apparatus for an endoscope comprising: a case; an operation member arranged outside the case; a pinion arranged within the case and rotationally driven by the operation member; and a pair of two rack members arranged so as to sandwich the pinion diametrically, meshing with the pinion, and connected to two operation wires arranged to extend up to a bending part arranged in a bendable fashion at a distal end of an insertion part inserted into a body to be examined, where
the operation wires are fixed at end portions thereof within the case, are arranged within tubular covering members arranged in the insertion part, and drive the bending part in a bending fashion by operation of the operation member, wherein
bend-preventing members which cover circumferences of portions of the operation wires included in the case in an extendable and contractable manner in driving directions of the operation wires to prevent bends of the operation wires are arranged, the portions of the operation wires being positioned between joint portions of the operation wires with the rack members and fixing portions thereof to the covering members.

2. The bending operation apparatus for an endoscope according to claim 1, wherein the bend-preventing members are coil-like members wound coarsely.

3. The bending operation apparatus for an endoscope according to claim 1, wherein the bend-preventing members are made of metal.

4. A bending operation apparatus for an endoscope comprising: a case; an operation member arranged outside the case; a pinion arranged within the case and rotationally driven by the operation member; and a pair of two rack members arranged so as to sandwich the pinion diametrically, meshing with the pinion, and connected to two operation wires arranged to extend up to a bending part arranged in a bendable fashion at a distal end of an insertion part inserted into a body to be examined, where
the operation wires are fixed at end portions thereof within the case, are arranged within tubular covering members arranged in the insertion part, and drive the bending part in a bending fashion by operation of the operation member, wherein
the bending operation apparatus for an endoscope further comprises a moving range setting means for determining moving ranges of the two rack members to the side of the insertion part collectively, the moving range setting means being disposed within the case such that a mounting position thereof can be changed.

5. The bending operation apparatus for an endoscope according to claim 4, wherein a plurality of pairs of operation wires for bending the bending part in different directions is provided, and the moving range setting means is arranged so as to correspond to each of the operations wires paired.

6. The bending operation apparatus for an endoscope according to claim 4, wherein the moving range setting means is screwed within the case such that a fixing position of the moving range setting means can be changed.

7. A bending operation apparatus for an endoscope comprising: a case; an operation member arranged outside the case; a pinion arranged within the case and rotationally driven by the operation member; and a pair of two rack members arranged so as to sandwich the pinion diametrically, meshing with the pinion, and connected to a pair of two operation wires arranged to extend up to a bending part arranged in a bendable fashion at a distal end of an insertion part inserted into a body to be examined, where
the operation wires are fixed at end portions thereof within the case, are arranged within tubular covering members arranged in the insertion part, and drive the bending part in a bending fashion by operation of the operation member, wherein
a cylindrical connection tube part is provided between the case and the insertion part, and a covering member insertion part for arranging the covering members along an inner face portion of the connection tube part is provided on the inner face portion of the connection tube part.

8. The bending operation apparatus for an endoscope according to claim 7, wherein the covering member insertion part is an annular member formed on the inner face portion of the connection tube part integrally with the connection tube part.

9. The bending operation apparatus for an endoscope according to claim 7, wherein the connection tube part is provided with a forceps insertion port for inserting a forceps into the insertion part.

10. An endoscope comprising: the bending operation apparatus for an endoscope according to any one of claims 1; and an observing means for observing inside of a body to be examined from the bending part.
